(19) United States
(12) Patent Application Publication
(10) Pub. No.: US 2010/0279388 A1
(43) Pub. Date: Nov. 4, 2010

(54) Agent for treating mildew and method for treating mildew

(75) Inventors: Keiko Sakamoto, Kumamoto (JP); Toshiaki Iwashita, Kumamoto (JP)

Correspondence Address: CHAPMAN AND CUTLER, 111 WEST MONROE STREET, CHICAGO, IL, 60603, US

(73) Assignee: Big Bio Co., Ltd., Kumamoto-shi ,Kumamoto (JP)

(21) Appl. No.: 12/733,598

(22) PCT Filed: Sep. 11, 2008
(86) PCT No.: PCT/JP2008/066445

§ 371 Date: Jun. 28, 2010

(30) Foreign Application Priority Data
Sep. 12, 2007 (JP) 2007-236665

(51) Int. Cl. C12N 1/20; A62D 3/02

(52) U.S. Cl. 435/252.5; 435/264

(57) ABSTRACT

It is intended to provide an agent for treating fungi which is capable of controlling the production cost while using a microorganism with high safety, and also maintaining an adhesion state on a surface of a target for a predetermined period of time or longer during use, and a method for treating fungi. The agent for treating fungi is characterized by containing a microorganism-containing liquid obtained by adding water to powder prepared by mixing a Bacillus sphaericus microorganism, a Bacillus subtilis microorganism, or a Bacillus thuringiensis microorganism with livestock manure treated at a high temperature between 60° C. and 150° C., a surfactant, and a propellant. The agent for treating fungi is characterized in that, preferably, the surfactant is at least one type selected from the group consisting of sodium lauryl sulfate, polyoxyethylene cetyl ether, and potassium fatty acid, and the propellant is LP gas.

### TECHNICAL FIELD

The present invention relates to an agent for treating mildew and a method for treating mildew, and in particular, to an agent for treating mildew and a method for treating mildew using a foam derived from a liquid containing a microorganism that is effective for treating mildew.

### BACKGROUND TECHNOLOGY

Humid, damp places where water is used a lot, such as bathrooms and kitchens in houses, easily attract mildew. Though conventionally, chlorine based or oxygen based bleach has been used to treat mildew, the bleach is mainly to make the color of mildew inconspicuous through its bleaching effects, and care is required to aerate the room sufficiently, as well as not to mistakenly use chlorine based bleach and acid detergents together.

Meanwhile, the present inventors found that the Bacillus sphaericus microorganism, the Bacillus subtilis microorganism and the Bacillus thuringiensis microorganism can be used for agents for preventing mildew and deodorants, as shown in Patent Document 1. In addition, these microorganisms are easily available from the soil, seawater, freshwater deposit, foods and the like, and it is shown that powders gained by mixing these microorganisms with livestock manure treated at a high temperature can be used to provide inexpensive agents for preventing mildew and deodorants.

Patent Document 5: Japanese Patent No. 3590019

In addition, Patent Document 1 also shows that water can be added to the gained powder, so that it can be used as a liquid. Such liquids make it possible for the microorganism to cling firmly to the object, so that it works effectively in comparison with when used as a powder. However, it is difficult to use powders and liquids on non-horizontal surfaces, such as walls, and it is difficult for the microorganism to keep clinging to smooth surfaces and water-repelling surfaces for a long period time.

### DISCLOSURE OF THE INVENTION

### Problem to Be Solved by the Invention

An object of the present invention is to solve the above described problem and provide an agent for treating mildew and a method for treating mildew using an utterly harmless microorganism, of which the cost of manufacture is low, and which clings to the surface of the object for a certain period of time when used.

### MEANS FOR SOLVING PROBLEM

As a result of diligent research, the present inventors found that the Bacillus sphaericus microorganism, the Bacillus subtilis microorganism and the Bacillus thuringiensis microorganism remain active as microorganisms for a long period of time, even in a state where a surfactant is added to a liquid including the microorganism, and furthermore the liquid includes a propellant and is contained in a spray can, for example, and thus completed the present invention.

The invention according to Claim 1 is an agent for treating mildew, characterized by having: a microorganism containing liquid gained by adding water to a mixed powder of Bacillus sphaericus, Bacillus subtilis or Bacillus thuringiensis and livestock manure treated at a high temperature of 60 degrees to 150 degrees; a surfactant; and a propellant.

The invention according to Claim 2 is the agent for treating mildew according to Claim 1, characterized in that the surfactant is at least one selected from the group consisting of sodium lauryl sulfate, polyoxyethylene cetyl ether and potassium fatty acid, and the propellant is an LP gas.

The invention according to Claim 3 is a method for treating mildew characterized in that a liquid for treating mildew has: a microorganism containing liquid gained by adding water to a mixed powder of Bacillus sphaericus, Bacillus subtilis or Bacillus thuringiensis and livestock manure treated at a high temperature of 60 degrees to 150 degrees; and a surfactant is foamed using a propellant, so that the liquid for treating mildew in the form of a foam clings to the object to be treated for mildew.

The invention according to Claim 4 is the method for treating mildew according to Claim 3, characterized in that the surfactant is at least one selected from the group consisting of sodium lauryl sulfate, polyoxyethylene cetyl ether and potassium fatty acid, and an LP gas is used as the propellant for the liquid for treating mildew when the liquid for treating mildew is foamed.

### EFFECTS OF THE INVENTION

The microorganism used in the present invention is Bacillus sphaericus, Bacillus subtilis or Bacillus thuringiensis, which are publicly known microorganisms. These microorganisms have been confirmed to be sufficiently harmless. Therefore, the agent for treating mildew according to the present invention is utterly harmless both during production and use, and has no harmful effects on the environment or human body. In addition, livestock manure treated at a high temperature of 60 degrees to 150 degrees is used as the nutrition source for the microorganism, and thus the material is available at extremely low cost, and in addition, livestock manure raises environmental issues, such as foul odor and pollution of rivers, and therefore, livestock manure can be used extremely effectively and beneficially.

Furthermore, according to the present invention, a microorganism containing liquid gained by adding water to a mixed powder of one of the above microorganisms and livestock manure treated at a high temperature is used. In addition, a surfactant is added to the microorganism containing liquid, and therefore, the liquid clings to the surface of the object to be treated for mildew in the form of a foam when used in a spray. As a result, the liquid for treating mildew remains clung for a certain period of time (for example several tens of minutes), and thus it become possible to effectively use the function of the microorganism on the surface of the object to be treated for mildew.

In addition, according to the present invention, the surfactant for foaming the liquid is at least one selected from the group consisting of sodium lauryl sulfate, polyoxyethylene cetyl ether and potassium fatty acid (may be pure soap, but preferably potassium fatty acid), and an LP gas is used as the propellant. All of these materials are used in articles for daily use, such as detergent, and have been confirmed to be safe for use.

### BEST MODE FOR CARRYING OUT THE INVENTION

The microorganism containing liquid used in the agent for treating mildew according to the present invention is gained by adding water to a mixed powder of Bacillus sphaericus, Bacillus subtilis or Bacillus thuringiensis and livestock manure treated at a high temperature of 60 degrees to 150 degrees. The amount of water added to the powder is not particularly limited, but usually approximately several tens of times more than the powder. The liquid gained by adding water to the powder is preserved at a predetermined temperature and aerated if necessary, so that the microorganism can be cultured. The microorganism containing liquid is filtered when used in the agent for treating mildew according to the present invention.

The microorganism used in the agent for treating mildew according to the present invention is Bacillus sphaericus, Bacillus subtilis or Bacillus thuringiensis, and these microorganisms are publicly known and easy to culture in soil, seawater, freshwater deposit and foods. In addition, these microorganisms are safe for the environment and the human body, and thus it is possible to provide agents for treating mildew using these as highly safe products.

Cow manure, pig manure and chicken manure are preferable as the livestock manure used in the present invention, and but it is also possible to use the manure of various other livestock, such as horse manure, if necessary. Livestock manure includes various bacteria, and high-temperature treatment of 60 degrees to 150 degrees is carried out for 5 hours, for example, in order to neutralize these bacteria. Livestock manure treated at a high temperature is dry and solid, and solid livestock manure is crushed and powdered, and the microorganism according to the present invention can be mixed with this powder.

It is necessary for the surfactant that is added to the microorganism containing liquid to allow Bacillus sphaericus, Bacillus subtilis or Bacillus thuringiensis to remain active over a long period of time (several months or more), and concretely, at least one selected from the group consisting of sodium lauryl sulfate, polyoxyethylene cetyl ether and potassium fatty acid is proper for use. The amount of surfactant added is not particularly limited, and approximately 0.1 to 10 in the case where the total volume of the microorganism containing liquid is 100 is preferable in order to further enhance the effects of the present invention.

The agent for treating mildew according to the present invention can be gained by injecting a liquid for treating mildew gained by adding a surfactant to a microorganism containing liquid into a spray can, which is then filled with an LP gas, which is a propellant. The propellant foams the agent for treating mildew when sprayed from the can, and there are no particular limitations, as long as it does not lower the activity of the microorganism in the can, or it makes it difficult for the activity of the microorganism to lower.

It has been confirmed that in the case where the surfactant is at least one selected from the group consisting of sodium lauryl sulfate, polyoxyethylene cetyl ether and potassium fatty acid, and the propellant is an LP gas, the microorganism remains active for approximately nine months.

Next, the method for treating mildew according to the present invention is described.

The above described agent for treating mildew is sprayed against the floor or walls to be treated for mildew, so that the liquid for treating mildew including the above described microorganism containing liquid and surfactant clings to the surface of the object to be treated for mildew in the form of a foam. When the foam is left for a predetermined period of time, for example several tens of minutes to several hours, the included microorganism effectively clings to the surface of the object to be treated for mildew, and thus, excellent mildew treating effects can be gained.

After a predetermined period of time, the foam is washed off with water. It is possible to use a neutral detergent or soap, if necessary, but it is necessary to prevent the activity of the microorganism clinging to the surface of the object to be treated for mildew from lowering, as well as to prevent the microorganism from being removed altogether.

In addition, it is also possible to remove the foam by wiping it off instead of washing it off with water.

Both while the foam is clinging to the object and after it is removed, it is possible for the microorganism on the surface of the object to be treated for mildew to kill the mildew or prevent further proliferation thereof. Examples

Next, experiments performed to confirm the working effects of the agent for treating mildew according to the present invention are described.

The agent for treating mildew according to the present invention was manufactured in the following procedure.

Livestock manure was treated at a high temperature of 60 ° C. to 150 ° C. for five hours and then crushed, and Bacillus sphaericus was added in such a ratio as to be 1 g to 10 g for ever 100 g of manure, which was then mixed. Water was added in such a ratio as to be 30 ml to every 1 g of the mixed powder, and the liquid was filtered after being cultured so that a microorganism containing liquid was manufactured.

One volume percent of a surfactant (sodium lauryl sulfate) was added to the total volume of the above described microorganism containing liquid so that a liquid for treating mildew was prepared.

90 g of a liquid for treating mildew was injected into an aluminum container having a volume of 100 ml, which was then filled with 6 g of an LP gas, which is a propellant, so that an agent for treating mildew was manufactured as a specimen.

Japan Food Research Laboratories were asked to carry out a test for the antimicrobial activity of the above described specimen of the agent for treating mildew.

In the test, the specimen was sprayed and left for a while, and a paper disc was impregnated with the thus gained liquid, and thus, a sample was gained. A test for the antimicrobial activity of the sample was carried out in reference to JIS L 19022002 “Method for Testing Antimicrobial Properties of Fiber Products/Antimicrobial Effects” 9. Qualitative Test (Halo Method). Here, Aspergillus niger IFO 6341 was used as a test bacteria.

A PDA medium (potato dextrose agar medium, produced by Eiken Chemical Co., Ltd.) and an SDA medium (Sabouraud's agar medium produced by Eiken Chemical Co., Ltd.) were used as test media.

In addition, the bacterial liquid, the sample and the plate medium for testing were prepared in accordance with the following methods.

(Preparation of Bacterial Liquid)

After the bacteria was cultured in a PDA medium at 25 ° C. +/- 1 ° C. for seven days, the formed spores were suspended in a saline solution to which 0.05% of polysorbate 80 was added so that the solution was prepared as a bacterial liquid with the number of spores per ml being approximately 10³.

(Preparation of Sample)

The specimen was sprayed and left for a while, and a paper disc having a diameter of approximately 10 ml was impregnated with 0.05 ml of the thus gained liquid, and thus, a sample was gained.

(Preparation of Plate Medium for Testing) 15 ml of an SDA medium was put into a dish and solidified. Furthermore, the dish was left at room temperature for 30 minutes so that the surface of the culture was dried, and thus, an agar plate medium was gained. A sample was placed on top of the agar plate medium and left at room temperature for 24 hours, and thus, a plate medium for testing was gained.

(Test Operation)

In the test operation, 0.5 ml of a bacterial liquid was sprayed onto a plate medium for testing so that the bacteria was cultured at 25 ° C. +/- 1 ° C. for seven days, and after that, whether or not there was a halo around the sample was determined through observation with the eye. Here, the number of living bacteria was measured in accordance with a poured plate culture method using an SDA medium (cultured at 25 ° C. +/- 1 ° C. for seven days).

The test results are shown in the following Table 1.

It can be seen from Table 1 that the agent for treating mildew according to the present invention has the effects of preventing further proliferation of mildew. In addition, the same effects of preventing further proliferation of mildew were confirmed when the same tests were carried out using polyoxyethylene cetyl ether or potassium fatty acid as the surfactant instead of sodium lauryl sulfate.

TABLE 1
Whether or not a Halo (Growth Preventing Band)
of a Sample is Formed Around the Test Bacteria

| | number of live bacteria in | |
|---|---|---|
| test bacteria | sprayed bacterial liquid (/ml) | results |
| Aspergillus niger | 1.8 × 10³ | +*¹ |
| IFO 6341 | | |

+: halo was observed
*¹the width of the halo was not measured due to inconsistency of the halo

### INDUSTRIAL APPLICABILITY

According to the present invention, it becomes possible to provide an agent for treating mildew using an utterly harmless microorganism, of which the cost of manufacture is low, and which clings to the surface of the object for a certain period of time when used, as well as a method for treating mildew.

1. An agent for treating mildew, comprising:
a microorganism-containing liquid gained by adding water to a mixed powder of Bacillus sphaericus, Bacillus subtilis or Bacillus thuringiensis, and livestock manure treated at a high temperature of 60 degrees C. to 150 degrees C.;
a surfactant; and
a propellant.

2. The agent for treating mildew according to Claim 1, wherein the surfactant is at least one selected from the group consisting of sodium lauryl sulfate, polyoxyethylene cetyl ether and potassium fatty acid, and the propellant is an LP gas.

3. A method for treating mildew, comprising the steps of:
making a microorganism-containing liquid by adding water to a mixed powder of Bacillus sphaericus, Bacillus subtilis or Bacillus thuringiensis, and livestock manure treated at a temperature of 60 degrees C. to 150 degrees C.;
making a foam by foaming said microorganism-containing liquid with a surfactant using a propellant; and
applying the foam to an object to be treated for mildew.

4. The method for treating mildew according to Claim 3, wherein the surfactant is at least one selected from the group consisting of sodium lauryl sulfate, polyoxyethylene cetyl ether and potassium fatty acid, and the propellant is an LP gas.
